SEQUENCE LISTING (3 sequences)

SEQ ID NO: 1 - DNA, 1282 bp, Escherichia coli
  source 1..1282: mol_type = genomic DNA
cgctgtaaggcgccagtagcgtttcacactgtttgactactgctgtgcctacgtctcgagataaaacgaaaggctcagtcgaaagactgggcctttcgttttataattgttagacattatttgccggctaccttggtgatctcgcccttcacgtagtggacaaattcttccaactgatctgcgcgcgaggccaagcgatcttcttcttgtccaagataagcctgtctagcttcaagtatgacgggctgatgctgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacgggatgatgtcgtcgtgcacaacaatggtgacttctacagcgcggagaatctcgctctctccaggggaagccgaagtttccaaaaggtcgttgatcaaagctcgccgcgttgtttcgtcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccagggcgtaacgcgcttgctgcttggatgcccgaggcatagactgtaccccaaaaaaacagtcatagcaagccatgaaaaccgccactgcgccgttaccaccgctgcgttcggccaaggttctggctcgagaaatcataaaaaatttatttgctttgtgagcggataacaattacaatagattcaattgtaagcggataacaatttcacacagaattcattaaagaggagaaattaactatggtttactcctataccgagaaaaaacgtattcgtaaggattttggtaaacgtccacaa

SEQ ID NO: 2 - DNA, 2526 bp, Escherichia coli
  source 1..2526: mol_type = genomic DNA
gtggcccgtatagcaggcattaacattcctgatcataagcatgccgtaatcgcattaacttcgatttatggcgtcggcaagacccgttctaaagccatcctggctgcagcgggtatcgctgaagatgttaagatcagtgagctgtctgaaggacaaatcgacacgctgcgtgacgaagttgccaaatttgtcgttgaaggtgatctgcgccgtgaaatcagcatgagcatcaagcgcctgatggatcttggttgctatcgcggtttgcgtcatcgtcgtggtctcccggttcgcggtcagcgtaccaagaccaacgcacgtacccgtaagggtccgcgcaaaccgatcaagaaataatcggggtgattgaataatggcaaaggcaccaattcgtgcacgtaaacgtgtaagaaaacaagtctctgacggcgtggctcatatccatgcttctttcaacaacaccatcgtgactatcactgatcgtcagggtaacgcgttgggttgggcaacagccggtggttccggtttccgtggttctcgcaaatccactccgtttgcagctcaggttgcagcagagcgttgcgctgacgccgtgaaagaatacggcatcaagaatctggaagttatggttaaaggtccgggtccaggccgcgaatctactattcgtgctctgaacgccgcaggtttccgcatcactaacattactgatgtgactccgatccctcataacggttgtcgtccgccgaaaaaacgtcgcgtataacgcctcgttttccaggtttgttggagaaagaaaatggcaagatatttgggtcctaagctcaagctgagccgtcgtgagggcaccgacttattccttaagtctggcgttcgcgcgatcgataccaagtgtaaaattgaacaagctcctggccagcacggtgcgcgtaaaccgcgtctgtctgactatggtgtgcagttgcgtgaaaagcaaaaagttcgccgtatctatggtgtgctggagcgtcagttccgtaactactacaaagaagcagcacgtctgaaaggcaacaccggtgaaaacctgttggctctgctggaaggtcgtctggacaacgttgtataccgtatgggcttcggtgccactcgtgcagaagcacgtcagctggttagccataaagcaattatggtaaacggtcgtgttgttaacatcgcttcttatcaggttagtccgaatgacgttgtaagcattcgtgagaaagcgaagaagcagtctcgcgtgaaagccgctctggagctggctgagcagcgtgaaaagccaacctggctggaagttgatgctggcaagatggaaggtacgtttaagcgtaagccggagcgttctgatctgtctgcggacattaacgaacacctgatcgtcgagctttactccaagtaaagcttagtaccaaagagaggacacaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaccggatcacaggttaaggttttactgagaaggataaggtcatgcgccatcgtaagagtggtcgtcaactgaaccgcaacagcagccatcgccaggctatgttccgcaatatggcaggttcactggttcgtcatgaaatcatcaagacgactctgcctaaagcgaaagagctgcgccgcgtagttgagccgctgattactcttgccaagactgatagcgttgctaatcgtcgtctggcattcgcccgtactcgtgataacgagatcgtggcaaaactgtttaacgaactgggcccgcgtttcgcgagccgtgccggtggttacactcgtattctgaagtgtggcttccgtgcaggcgacaacgcgccgatggcttacatcgagctggttgatcgttcagagaaagcagaagctgctgcagagtaa

SEQ ID NO: 3 - DNA, 4500 bp, Escherichia coli
  source 1..4500: mol_type = genomic DNA
atgcaaaagtttgataccaggaccttccagggcttgatcctgaccttacaggattactgggctcgccagggctgcaccattgttcaaccattggacatggaagtcggcgcgggaacctctcacccaatgacctgtctgcgcgagctggggccagaaccgatggcggctgcttatgttcagccttctcgtcgcccgaccgatggtcgctacggcgaaaaccccaaccgtttacagcactactatcagttccaggtggtcattaagccatcgccggacaatattcaggagctgtacctcggttctctgaaagagctgggcatggacccgactattcacgacatccgtttcgtggaagataactgggaaaacccgacgctgggtgcctggggactgggctgggaagtgtggctgaacggcatggaagtgacgcagttcacttacttccagcaggttggtggtctggagtgtaaaccggttaccggcgagatcacctacggtctggaacgtctggccatgtacattcagggcgtagacagcgtttacgacctggtctggagcgacggcccgctgggtaaaaccacctacggcgacgtgttccatcagaacgaagtggagcagtccacttacaacttcgaatacgcggatgtggacttcctgttcacctgcttcgagcagtacgagaaagaagcgcagcagctgctggcgctggaaaatccgctgccgctgccagcctacgagcgtattctgaaagccgcccacagcttcaacctgctggatgcgcgtaaagccatctccgtcaccgagcgtcagcgctacattctgcgcattcgcaccctgaccaaagcagtggcagaagcatactacgcttcccgtgaagccctcggcttcccgatgtgcaacaaagataagtaagaggcggctatgtctgagaaaacttttctggtggaaatcggcactgaagagctgccaccaaaagcactgcgcagcctggctgagtcctttgctgcgaactttactgcggagctggataacgctggcctcgcacacggcaccgttcaatggtttgctgctccgcgtcgtctggcgctgaaagtagctaacctggcggaagcgcaaccggatcgtgaaatcgaaaaacgcggcccggcgattgcccaggcgttcgacgctgaaggcaaaccgagcaaagcggcagaaggttgggcgcgtggttgcggtattaccgttgaccaggctgagcgtctgactaccgataaaggcgaatggctgctgtatcgcgcccatgtgaagggcgaaagcaccgaagcactgctgccgaatatggttgcgacttctctggcgaaactgccgatcccgaaactgatgcgttggggcgcaagcgacgtgcacttcgtgcgtccggtgcacaccgtgaccctgctgctgggcgacaaagtcattccggcaaccattctgggcattcagtccgatcgcgtgattcgcggccaccgctttatgggcgagccggaattcaccatcgataacgccgatcagtatccggaaattctgcgtgagcgtgggaaagtcatcgccgattacgaagaacgtaaggcgaagattaaagccgatgccgaagaagcagcgcgtaagattggcggtaacgctgacttaagcgaaagcctgctggaagaagtggcttcgctggtggagtggccggtcgttctgaccgcaaaattcgaagagaaattcctcgcggtgccggctgaagcgctggtttacaccatgaaaggtgaccagaaatacttcccggtgtatgcgaacgacggcaaactgctgccgaactttatcttcgttgccaacatcgaatcgaaagatccgcagcagattatctccggtaacgagaaagtcgttcgtccgcgtctggcggatgccgagttcttcttcaacaccgaccgtaaaaaacgtcttgaagataacctgccgcgcctgcaaaccgtgttgttccagcaacagttggggacgctgcgcgacaaaactgaccgcatccaggcgctggctggctggattgctgaacagattggcgctgacgttaaccacgctacccgtgcgggtctgctgtctaagtgcgacctgatgaccaacatggtcttcgagttcaccgacacccagggcgttatggggatgcactatgcgcgtcacgatggcgaagcggaagatgtcgcggtggcgctgaatgagcagtatcagccgcgttttgctggtgatgacctgccgtccaacccagtagcttgtgcgctggcgattgctgacaagatggataccctggcgggtatcttcggtatcggtcagcatccgaaaggcgacaaagacccgtttgcgctgcgtcgtgccgcgcttggcgtgctgcgaattatcgttgagaagaacctcaaccttgatctgcaaacgctgaccgaagaagcggtgcgtctgtatggcgataagctgactaatgccaacgtagttgatgatgttatcgactttatgctcggtcgcttccgcgcctggtatcaggacgaaggttataccgttgacaccatccaggcggtactggcgcgtcgtccgactcgtccggctgatttcgatgcccgtatgaaagcggtatcgcatttccgtaccctggatgcagctgctgcactggcggcggcgaacaaacgtgtatctaacattctggcgaaatctgacgaagtgctgagcgaccgcgtgaatgcctctaccctgaaagagccggaagaaattaaactggcgatgcaggttgtggtgctacgtgacaagctggagccgtactttacggaaggtcgttaccaggatgcgctggtcgaactggctgagctgcgtgaaccggttgatgctttcttcgataaagtgatggtcatggttgatgacaaagaattgcgtatcaaccgtctgaccatgctggagaaactgcgcgaactgttcctgcgcgttgcggatatttcgctgttgcaataataacgccgttattaaatagcctgccatctggcaggctttttttatcgctaaataatacagcaacctttaataatcttctgctgaataaagagaaatcataaaaaatttatttgctttgtgagcggataacaattacaatagattcaattgtaagcggataacaatttcacacagaattcattaaagaggagaaattaactatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagagttcgatccgatcctgctgcgccctgttgacgatctggaattgactgtccgctctgctaactgccttaaagcagaagctatccactatatcggtgatctggtacagcgtaccgaggttgagctccttaaaacgcctaaccttggtaaaaaatctcttactgagattaaagacgtgctggcttcccgtggactgtctctgggcatgcgcctggaaaactggccaccggcaagcatcgctgacgagtaaataataatagattttattacgcgatcgattatttatttcctgaaaacaaataaaaaaatccccgccaaatggcagggatcttagattctgtgcttttaagcagagattacaggctggttacgttaccagctgccgggcctttagcgccgctttcgatggtgaaggacactttctgaccttcgtccagagatttgtaaccatcgttctggatagcagagaagtgtacgaacacatctttagagccatcgtcaggagtgatgaagccgaagcctttgtcagcgttgaaccattttacgataccagtcattttaccggacat

(19) United States
(12) Patent Application Publication
(10) Pub. No.: US 2026/0176664 A1
(43) Pub. Date: Jun. 25, 2026

(54) A genetically engineered bacterium and genetically stable method for the production of metabolites, peptides, and-or recombinant proteins

(71) Applicant: INSTITUT NATIONAL DE RECHERCHE EN INFORMATIQUE ET EN AUTOMATIQUE, Le Chesnay (FR)

(72) Inventors: Hidde De Jong, Le Chesnay (FR); Johannes Geiselmann, Le Chesnay (FR); Thibault Clavier, Le Chesnay (FR); Célia Boyat, Le Chesnay (FR)

(21) Appl. No.: 19/126,441

(22) PCT Filed: Oct. 30, 2023
(86) PCT No.: PCT/EP2023/080253

§ 371 Date: May 1, 2025

(30) Foreign Application Priority Data
Nov. 2, 2022 (FR) FR2211392

(51) Int. Cl. C12P 21/00; C12N 9/12; C12P 1/04

(52) U.S. Cl. CPC C12P 21/00; C12N 9/1247; C12P 1/04; C12Y 207/07006

(57) ABSTRACT

The invention concerns a genetically engineered bacterium for producing at least one metabolite, peptide or recombinant protein comprising a gene encoding a recombinant protein of interest or at least one gene encoding an enzyme involved in the production of a peptide or metabolite of interest. The invention is further directed to methods using the bacterium for producing at least one metabolite, peptide or recombinant protein.

The present invention is relative to a genetically engineered bacterium capable of producing at least one metabolite, peptide or recombinant protein and to a method for the bioproduction of at least one metabolite, peptide or recombinant protein.

In a more general aspect, the present invention is relative to applications of synthetic biology in metabolic engineering, for the production of metabolites, peptides, and heterologous proteins of biotechnological or medical interest.

Many microorganisms are able to synthetize compounds, such as biofuels, bulk and fine chemicals, or molecules for medical applications. The modification of such microorganisms in order to optimize or maximize the production of the desired compounds is one of the major challenges in the field of biotechnology.

Genetic engineering allows the redesign of the metabolic flux in microorganisms or more generally in cells. In particular, modified cells of microorganism are ideally able to switch from growth (i.e. biomass production), to product synthesis (i.e. production of a compound of interest). Accordingly, industrial processes generally comprise two major steps: a first step in which a cellular population is grown to a desired size and in which the target compound of interest is parallelly produced, and a second step in which cellular growth is shut down to enable the cells to produce the target compound without using resources otherwise needed for cell growth. During the first step most of the available energy and resources are used for biomass formation. As a consequence, the yield of the compound of interest is small during that first step. However, inhibiting cellular growth uncouples the production of the target compound from biomass production. In other words, when cellular growth is shut down, the microorganism cells can concentrate on producing the target compound; cf. Chotani et al. (2000) Biochim. Biophys. Acta 1543:434-455 and Sonderegger M, Schüperli M, Sauer U. 2005. Selection of quiescent Escherichia coli with high metabolic activity. Metab Eng 7:4-9.

Generally, growth arrest occurs spontaneously when the cell density reaches a certain limit value. Yet, when the cell density is too high it is generally accompanied by a high level of cell morbidity and a severe reduction of metabolic activity in the cells. This has a negative effect on the industrial process since it drastically decreases productivity of the target compound.

This is why biotechnological processes, especially on an industrial scale, need to control bacterial growth. The possibility to externally control bacterial growth is essential for many applications in metabolic engineering, where the objective is to simultaneously optimize titer, productivity, and yield; cf. Stephanopoulos G. 2012, Synthetic biology and metabolic engineering, ACS Synth Biol 1:514-525 and Van Dien S. 2013, From the first drop to the first truckload: Commercialization of microbial processes for renewable chemicals, Curr Opin Biotechnol 24:1061-1068. Classical approaches for growth control are based on the responses of bacteria to temperature, antibiotics, or nutrient limitation Sonderegger M, Schüperli M, Sauer U. 2005, Selection of quiescent Escherichia coli with high metabolic activity, Metab Eng 7:4-9; Tunner J R, Robertson C R, Schippa S, Matin A. 1992, Use of glucose starvation to limit growth and induce protein production in Escherichia coli, Biotechnol Bioeng 40:271-279; Li S, Jendresen C B, Nielsen A T. 2016, Increasing production yield of tyrosine and mevalonate through inhibition of biomass formation, Process Biochem 51:1992-2000; Rowe D C, Summers D K. 1999, The quiescent-cell expression system for protein synthesis in Escherichia coli, Appl Environ Microbiol 65:2710-2715; Harder B J, Bettenbrock K, Klamt S. 2018, Temperature-dependent dynamic control of the TCA cycle increases volumetric productivity of itaconic acid production by Escherichia coli, Biotechnol Bioeng 115:156-164; and Schramm T, Lempp M, Beuter D, Sierra S G, Glatter T, Link H. 2020, High-throughput enrichment of temperature-sensitive argininosuccinate synthetase for two-stage citrulline production in E. coli, Metab Eng 16:14-24.

Over the past decade, synthetic biology has enriched the available technologies for bacterial growth control. New technologies include genome and plasmid engineering (cf. Kosuri S, Church G. 2014, Large-scale de novo DNA synthesis: technologies and applications, Nat Methods 11:499-507, and Csörgö B, Nyerges Á, Pósfai G, Fehér T. 2016, System-level genome editing in microbes, Curr Opin Microbiol 33:113-122), the computer-aided design and construction of genetic circuits (cf. Nielsen A A K, Der B S, Shin J, Vaidyanathan P. Paralanov V, Strychalski E A, Ross D, Densmore D, Voigt C A. 2016, Genetic circuit design automation, Science (80-) 352:aac7341), optogenetic control of gene expression (cf. Olson E J, Tabor J J. 2014, Optogenetic characterization methods overcome key challenges in synthetic and systems biology, Nat Chem Biol 10:502-511), and fluorescent reporters for live monitoring of gene expression (cf. Zaslaver A, Bren A, Ronen M, Itzkovitz S, Kikoin I, Shavit S, Liebermeister W, Surette M G, Alon U. 2006, A comprehensive library of fluorescent transcriptional reporters for Escherichia coli, Nat Methods 3:623-628), enabling the feedback control of cellular processes. Indeed, synthetic biology approaches towards growth control have exploited feedback control of genes responsible for amino acid synthesis (cf. Milias-Argeitis A, Rullan M, Aoki S K, Buchmann P, Khammash M. 2016, Automated optogenetic feedback control for precise and robust regulation of gene expression and cell growth, Nat Commun 7:12546), competition between host RNA polymerase and the phage T7 RNA polymerase (cf. Stargardt P, Feuchtenhofer L, Cserjan-Puschmann M, Striedner G, Mairhofer J. 2020, Bacteriophage inspired growth-decoupled recombinant protein production in Escherichia coli, ACS Synth Biol 9:1336-1348), feedback control of mRNA decay (cf. Venturelli O S, Tei M, Bauer S, Chan LJG, Petzold C J, Arkin A P. 2017, Programming mRNA decay to modulate synthetic circuit resource allocation, Nat Commun 8:15128), and a toggle switch for decoupling growth and lactate production (cf. Venayak N, Raj K, Jaydeep R, Mahadevan R. 2018, An optimized bistable metabolic switch to decouple phenotypic states during anaerobic fermentation, ACS Synth Biol 7:2854-2866).

The Applicant of the present invention has previously developed a radically different approach for bacterial growth control which is based on a so-called synthetic growth switch. Details of the techniques implicated in that synthetic growth switch are disclosed in EP 3 047 031, and further in Izard J, Gomez Balderas C, Ropers D, Lacour S, Song X, Yang Y, Lindner A B, Geiselmann J, De Jong H. 2015, A synthetic growth switch based on controlled expression of RNA polymerase, Mol Syst Biol 11:840. In short, a synthetic growth switch enables the control of cell growth by means of controlling the expression of genes coding for RNA polymerase by specific chemical compounds. This technique thus involves growth-related genes that can be selectively expressed or repressed by feeding a chemical compound to the microorganisms. In other words, the expression or the repression of the mutated gene is dependent on the presence or absence of the chemical compound.

Yet, synthetic growth switches are fragile by nature. This is mainly due to the effect that cells tend to accumulate mutations in the system responsible for the inhibition of RNA polymerase, thus favoring growth. Consequently, synthetic growth switches systems are often rendered inoperative by spontaneous mutations.

There is a need to provide a genetically modified bacterium with improved genetic stability while preserving its metabolic production capacity.

To that end, the object of the invention is a genetically engineered bacterium for producing at least one metabolite, peptide or recombinant protein comprising a gene encoding a recombinant protein of interest or at least one gene encoding an enzyme involved in the production of a peptide or metabolite of interest, and further comprising genes encoding the ββ′ subunits of an RNA polymerase operably linked to an inducible promoter, the natural promoter of said genes being replaced by said inducible promoter, characterized in that:
either it further comprises (according to a first embodiment) a gene encoding the α subunit of the RNA polymerase having the following features listed below:
   said gene being operably linked to a second inducible promoter, the natural promoter of the gene being replaced by said second inducible promoter,
   said gene being inserted by homologous recombination into a locus constituted of genes that are non-essential for the growth of the bacteria (in other terms that does not contain genes involved in the growth of bacteria) or into a non-coding sequence and said locus not being subject to spontaneous homologous recombination,
   said gene encoding the α subunit of the RNA polymerase being positioned at a maximum distance of 500,000 nucleotides from the replication origin, and
   said gene being oriented in the opposite direction of the upstream gene,
and the natural gene encoding the α subunit of the RNA polymerase is deleted from its natural location,
or it further comprises (according to a second embodiment) a least one gene encoding at least one small regulatory RNA (srRNA) inhibiting the translation of messenger RNAs from the genes encoding the ββ′ subunits of RNA polymerase so as to inhibit the synthesis of ββ′ subunits of RNA polymerase, said gene encoding small regulatory RNA being operably linked to a second inducible promoter.

In the present description, “a locus is not subject to spontaneous homologous recombination” means a gene sequence whose probability of being the subject of spontaneous natural homologous recombination is less than 30%, preferably less than 20%, even more preferably less than 10%.

These different dual mechanisms for controlling gene expression permit to increase the stability of the growth change of the bacteria. In fact, even in the event of a mutation in one of the inducible promoter regions, these mechanisms enable the bacteria's growth to continue to be controlled.

In particular, in the case of the first embodiment, the additional control of the transcription of the α subunit increases stability of growth control.

It should be noted that, when the gene coding for the subunit is not displaced, it is not possible to obtain genetically stable bacteria. This is due to the fact that the gene encoding the α subunit of the RNA polymerase is located in the middle of an operon comprising ribosomal genes. If the gene encoding the α subunit of the RNA polymerase is not displaced, the regulation of the operon may be disrupted, interfering with the expression of ribosomal genes which are involved in the translation process.

The same failure is obtained when the above-mentioned insertion criteria are not respected.

Advantageously, when the first and second promoters are of the same type, the expression of the α subunit and ββ′ subunits of the RNA polymerase can be regulated at the same time.

In particular, in the case of the second embodiment, the additional and indirect control of the translation of the mRNAs of ββ′ subunits by the snRNA allows to increase stability of growth control. This construction is particularly interesting in that it allows growth control at the transcription level as well as translation level.

In this second embodiment, there may be one or more srRNAs targeting messenger RNAs of the β or β′ subunits. When only one type of srRNA is produced, it is preferentially designed to inhibit translation of the RNA encoding the β subunit.

Advantageously, in these embodiments, when the first and second promoters are different in nature, the expression of the subunits and srRNAs can be regulated precisely and simultaneously, allowing to control precisely growth.

According to a variant of the first embodiment, the encoding of the α subunit of the RNA polymerase operably linked to a second inducible promoter is inserted by homologous recombination into one of the following loci: insG, yjhD, insO-insl1-insM, insA-insB1, ryjB, yjhR, envZ-ompR, arcZ, or sokA-insKJ-hokA.

According to a variant of these embodiments, said first inducible promoter and said second inducible promoter are two distinct types of inducible promoters.

According to a variant of these embodiments, at least one of said inducible promoters depend on two distinct external inducer molecules.

According to a variant of these embodiments, at least one of said inducible promoters is selected from the following promoters: IPTG-dependent promoter, arabinose-dependent promoter (such as AraC), rhamnose-dependent promoter.

According to a variant of the first embodiment, the genes encoding the ββ′ subunits of RNA polymerase are the rpoBC genes of the rpoBC operon and the gene encoding the α subunit of RNA polymerase is the rpoA gene of the rpsM operon.

According to a variant of these embodiments, the bacterium is of the species Escherichia coli.

According to a variant of these embodiments, wherein the original rpoA locus is replaced by a selection marker, preferentially a selection marker conferring chloramphenicol resistance (cmR), by homologous recombination.

According to a variant of these embodiments, the bacterium comprises at least three copies of the lacI gene.

According to a variant of the first embodiment, the bacterium of the invention comprises a nucleotide sequence having at least 80%, preferentially 90%, and more preferentially 100% identity to at least one nucleotide sequence from the group consisting of SEQ ID NO: 1, SEQ ID NO: 2 and SEQ ID NO: 3

According to a variant of the first embodiment, the bacterium of the invention comprises a nucleotide sequence having at least 80%, preferentially 90%, and more preferentially 100% identity to SEQ ID: 1, SEQ ID: 2 and SEQ ID: 3.

Another object of the present invention is a method (of the first embodiment) for producing at least one metabolite, peptide or recombinant protein of interest comprising the steps of
a. culturing bacteria comprising: (i) a gene encoding said recombinant protein or at least one gene encoding an enzyme involved in the production of said peptide or metabolite, (ii) genes encoding the ββ′ subunits of bacterial RNA polymerase operably linked to a first inducible promoter, the natural promoter of said genes being replaced by said first inducible promoter, and (iii) a gene encoding the α subunit of bacterial RNA polymerase in a first culture medium inducing the expression of the genes encoding the ββ′ subunits and further inducing the expression of the gene encoding the α subunit, thereby inducing bacterial growth, the α subunit of bacterial RNA polymerase having the following features: said gene being operably linked to a second inducible promoter, the natural promoter of the gene being replaced by said second inducible promoter, said gene being inserted by homologous recombination into a locus constituted of genes that are non-essential for the growth of the bacteria or into a non-coding sequence and said locus not being subject to spontaneous homologous recombination, said gene encoding the α subunit of the RNA polymerase being positioned at a maximum distance of 500,000 nucleotides from the replication origin, and said gene being oriented in the opposite direction of the upstream gene, and the natural gene encoding the α subunit of the RNA polymerase is deleted from its natural location;
b. culturing said bacteria in a second culture medium inhibiting the expression of the genes encoding the ββ′ subunits and further inhibiting the expression of the gene encoding the α subunit, thereby producing said metabolite, peptide or recombinant protein while inhibiting bacterial growth;
c. optionally iterating steps a. and b. successively; and
d. optionally recovering said metabolite, peptide or recombinant protein produced by said bacteria.

According to a variant of the first embodiment, each inducible promoter is an IPTG-dependent promoter or a lactose-dependent promoter.

According to a variant of the first embodiment, the first culture medium of step a. comprises IPTG or lactose and the second culture medium of step b. is free of IPTG or lactose.

Another object of the present invention is a method (of the second embodiment) for producing at least one metabolite, peptide or recombinant protein of interest comprising the steps of
a. culturing bacteria comprising: (i) a gene encoding said recombinant protein or at least one gene encoding an enzyme involved in the production of said peptide or metabolite, (ii) genes encoding the ββ′ subunits of bacterial RNA polymerase operably linked to a first inducible promoter, the natural promoter of said genes being replaced by said first inducible promoter, and (iii) at least one gene encoding at least one small regulatory RNA inhibiting the translation of messenger RNAs from the genes encoding the ββ′ subunits of RNA polymerase so as to inhibit the synthesis of ββ′ subunits of RNA polymerase, said gene encoding small regulatory RNA being operably linked to a second inducible promoter, in a first culture medium inducing the expression of the genes encoding the ββ′ subunits and inhibiting the expression of the gene encoding the small regulatory RNA, thereby inducing bacterial growth;
b. culturing said bacteria in a second culture medium inhibiting the expression of the genes encoding the ββ′ subunits and further inducing the expression of the gene encoding the small regulatory RNA, thereby producing said metabolite, peptide or recombinant protein while inhibiting bacterial growth;
c. optionally iterating steps a. and b. successively; and
d. optionally recovering said metabolite, peptide or recombinant protein produced by said bacteria.

According to a variant of these embodiments, the gene encoding said recombinant protein or said at least one gene encoding an enzyme involved in the production of said peptide or metabolite is transcribed by a second RNA polymerase having a catalytic subunit or catalytic subunits that are different from the ββ′ subunits and the α subunit of the RNA polymerase operably linked to the inducible promoter.

According to a variant of these embodiments, the second RNA polymerase is the bacteriophage T7 polymerase.

According to a variant of these embodiments, the bacteria are Escherichia coli bacteria.

Other features and advantages of the embodiments according to the invention will stand out and/or become clear upon reading the following description, which comprises specific examples given in an illustrative and non-limiting manner, as well as from the drawings in which:

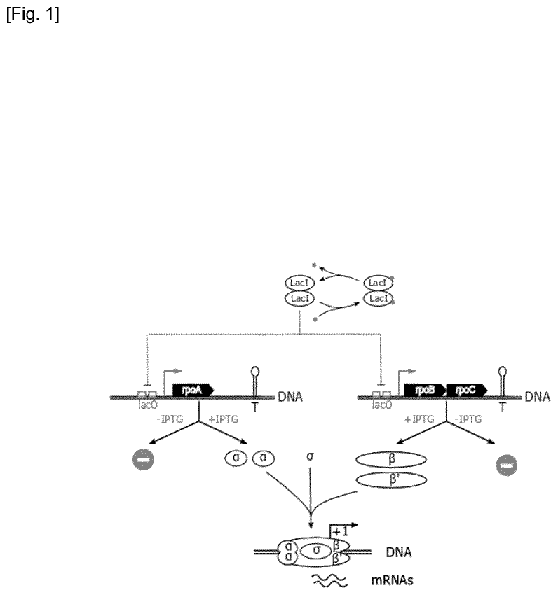

FIG. 1 shows a conceptual view of an embodiment according to the invention;

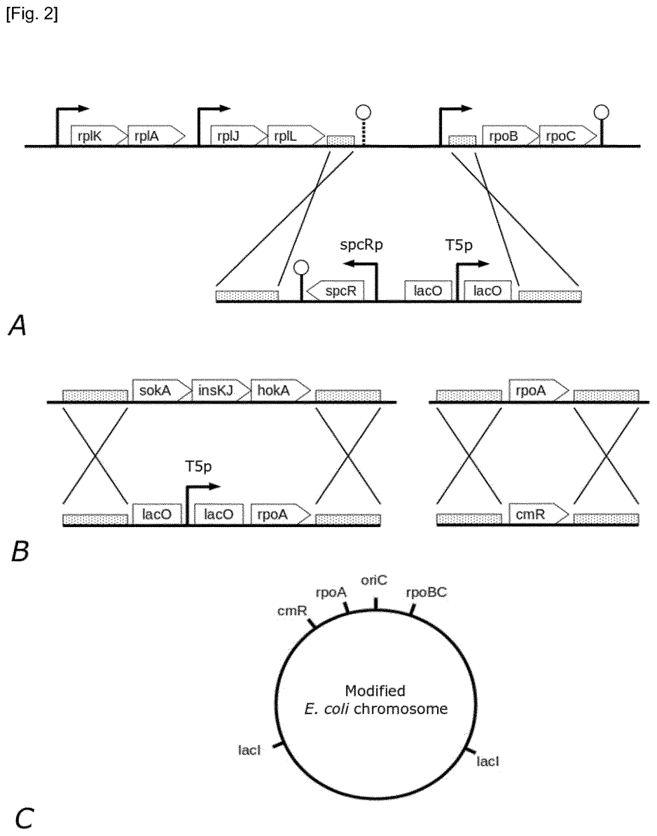

FIG. 2 shows the genetic construction for IPTG-dependent control of rpoBC and rpoA transcription of a variant according to the invention;

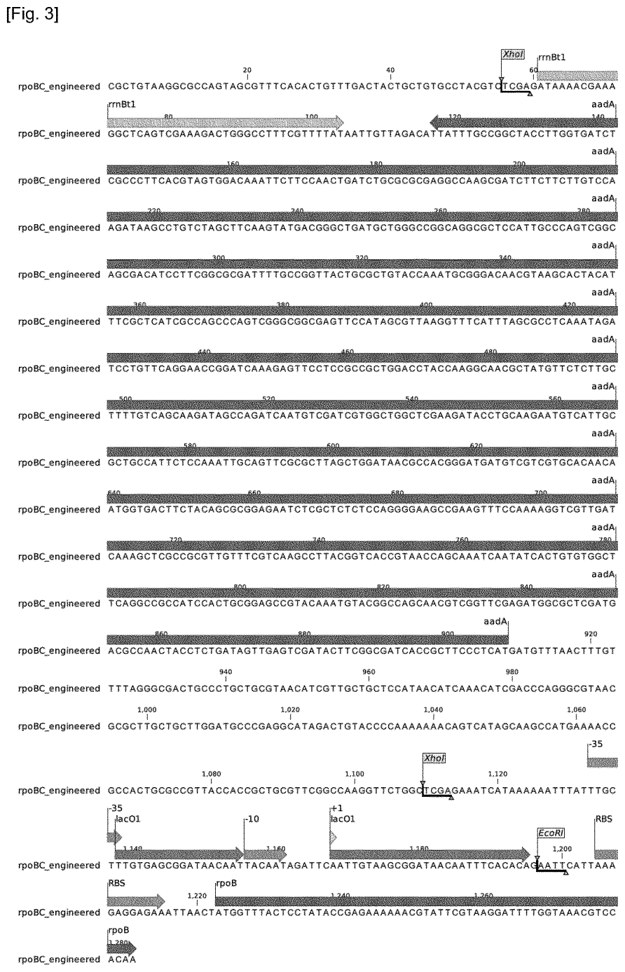

FIG. 3 shows a sequence according to a variant of the invention wherein rpoBC is under the control of an IPTG dependent promoter;

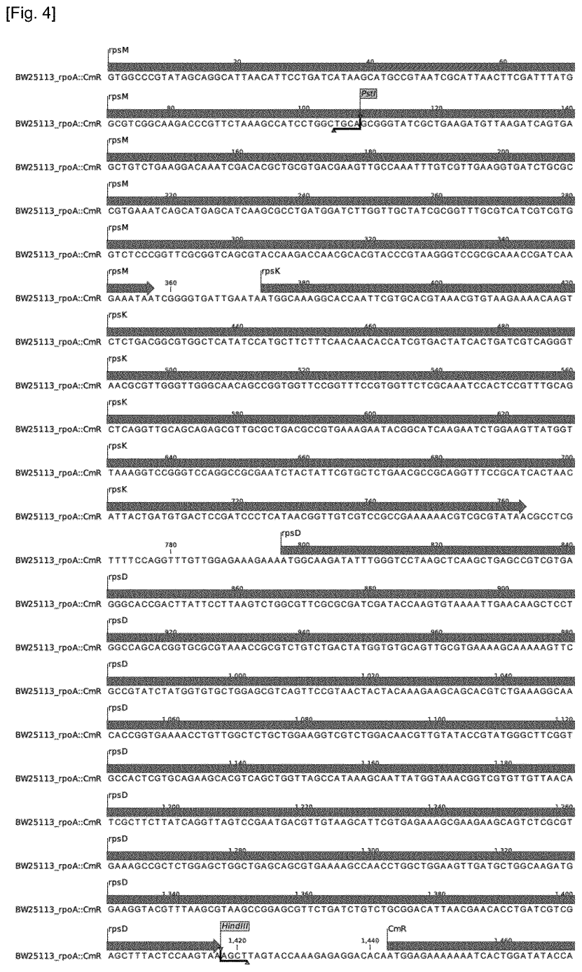

FIG. 4 shows a first part of a sequence according to a variant of the invention wherein rpoA is replaced by Cm in the operon of ribosomal proteins starting with rpsM;

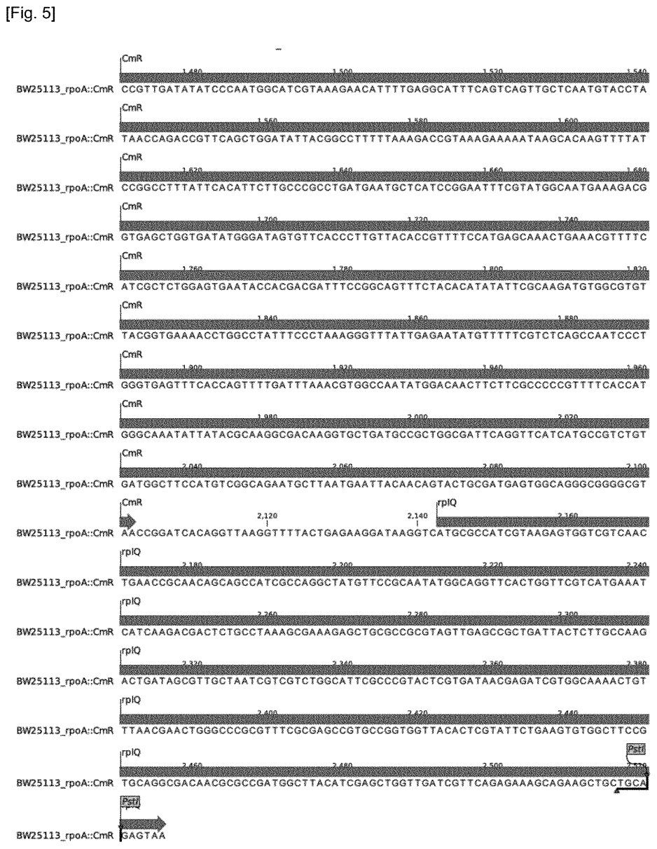

FIG. 5 shows a second part of the sequence of FIG. 4;

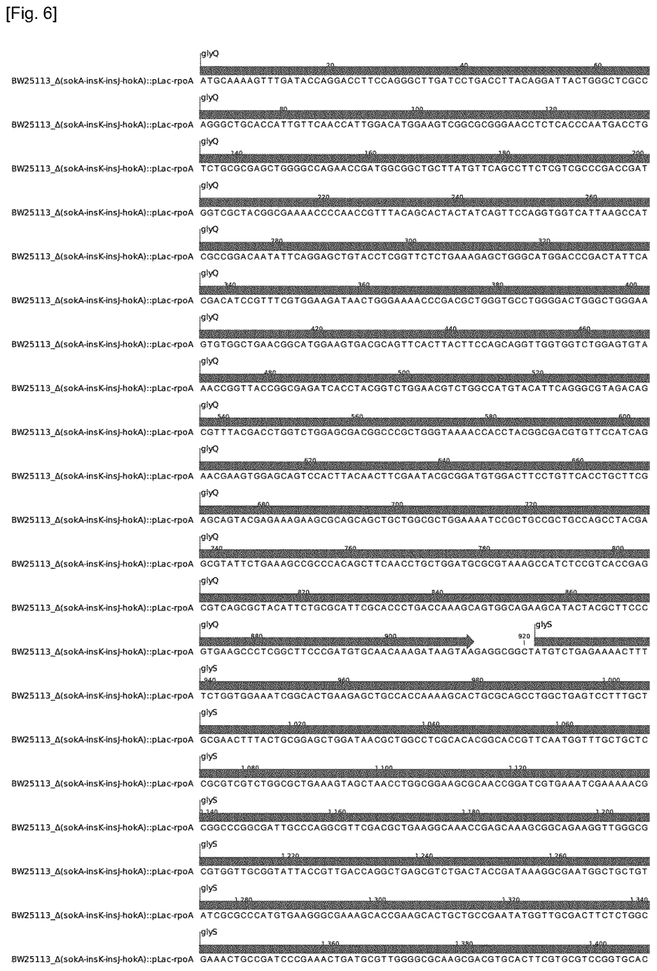

FIG. 6 shows a first part of a sequence according to a variant of the invention wherein rpoA, which is under the control of an IPTG-dependent promoter;

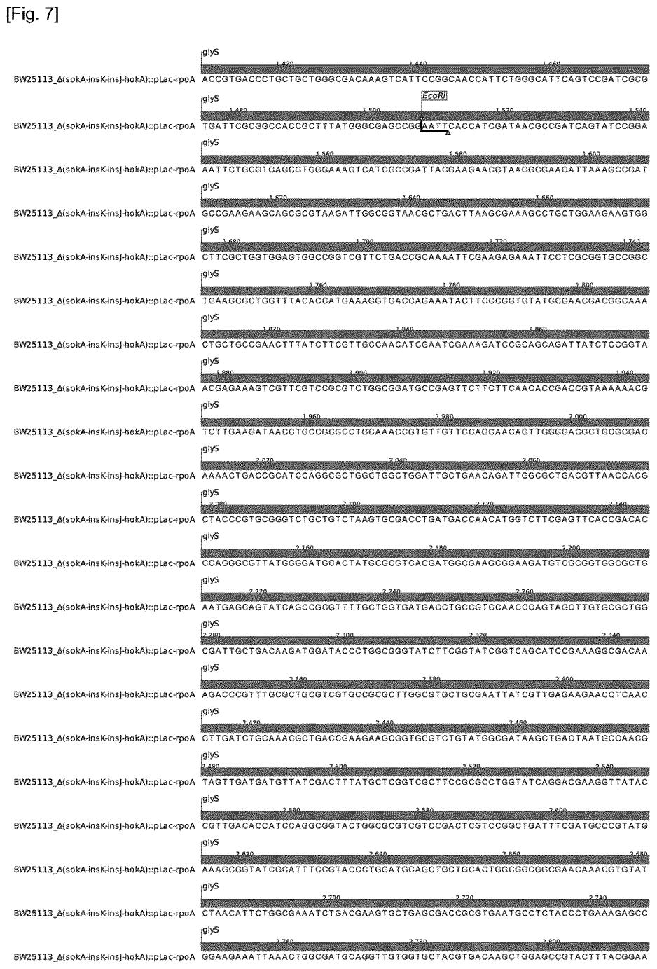

FIG. 7 shows a second part of the sequence of FIG. 6;

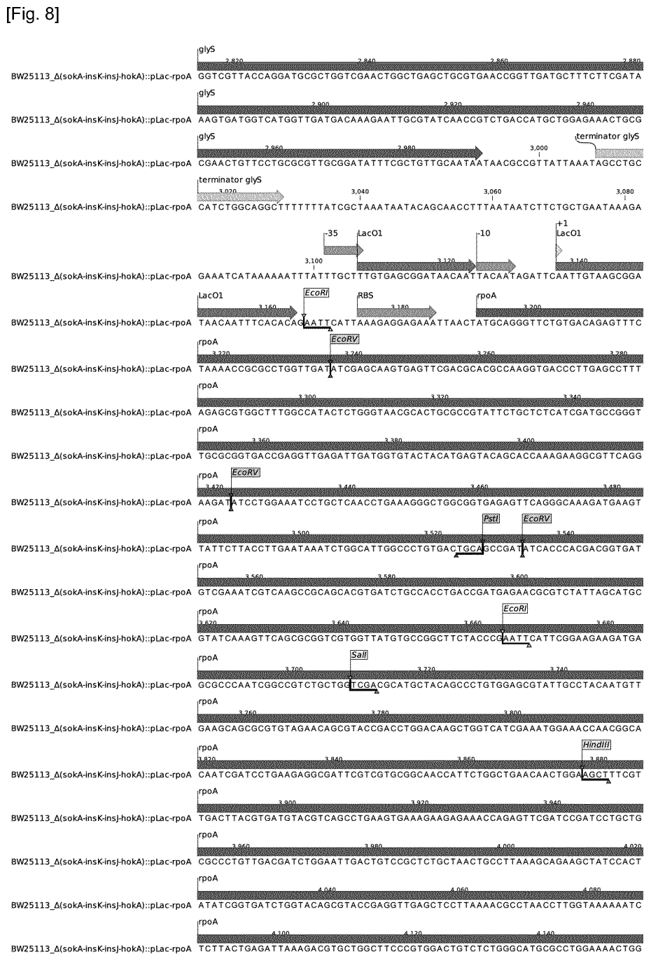

FIG. 8 shows a third part of the sequence of FIG. 6;

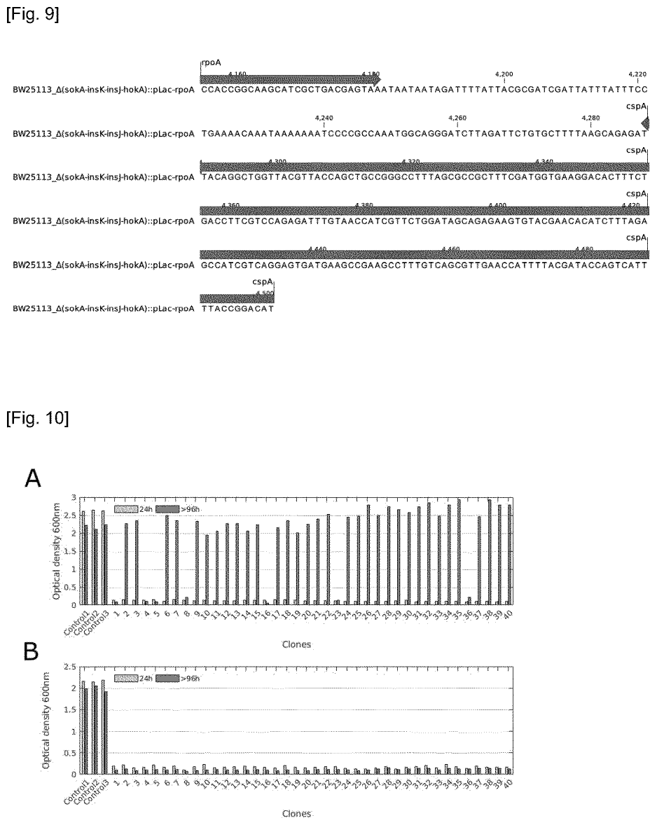

FIG. 9 shows a fourth part of the sequence of FIG. 6;

FIG. 10 shows the increased genetic stability of a bacterial strain according to a variant of the invention;

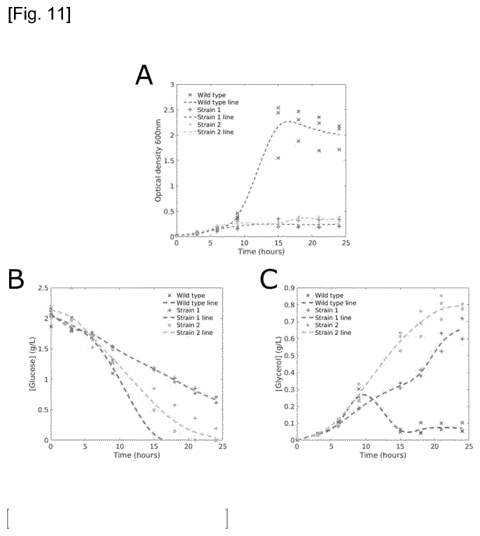

FIG. 11 shows growth, glucose consumption, and glycerol production of a wild-type strain and the strains with a simple and a double control of RNA polymerase expression;

FIG. 12A shows the expected genomic region after integration of sequence with inducible rpoA gene into the yhim-yhiN locus;

FIG. 12B shows the results of agarose gel migration of DNA fragments obtained under different PCR conditions; and

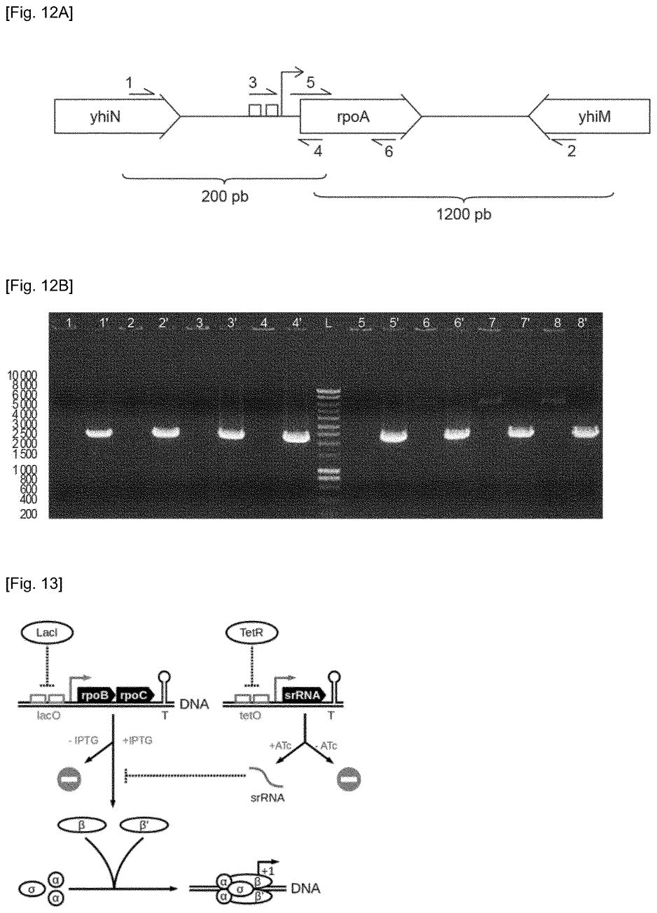

FIG. 13 shows a conceptual view of another embodiment according to the invention.

The drawings and the description herein contain, for the most part, elements of definite nature. Therefore, description and drawings not only are being used to better understand the embodiments of the invention, but also to contribute to the definition thereof, when appropriate.

The Applicant previously engineered a bacterial strain of the species Escherichia coli where the transcription of a key component of the gene expression machinery, RNA polymerase, is under the control of an inducible promoter, cf. EP 3 047 031. By changing the inducer concentration in the medium, it is possible to adjust the RNA polymerase concentration and thereby switch bacterial growth between zero and the maximal growth rate supported by the medium. The Applicant endowed both the wild-type E. coli strain and the modified strain of the invention with the capacity to produce glycerol when growing on glucose. Cells in which growth has been switched off continue to be metabolically active and produce glycerol at a twofold higher yield than in cells with natural control of RNA polymerase expression. The resulting yield was shown to be close to the theoretical maximum; cf. Izard J, Gomez Balderas C, Ropers D, Lacour S, Song X, Yang Y, Lindner A B, Geiselmann J, De Jong H. 2015, A synthetic growth switch based on controlled expression of RNA polymerase, Mol Syst Biol 11:840. The synthetic switch thus separates growth from target compound production. This approach is optimal from a control-theoretical point of view (cf. Yegorov I, Mairet F, de Jong H, Gouzé J L. 2019, Optimal control of bacterial growth for the maximization of metabolite production, J Math Biol 78:985-1032), and that has been generally adopted in the art, cf. Lo T M, Chng S H, Teo W S, Cho H S, Chang M W. 2016, A two-layer gene circuit for decoupling cell growth from metabolite production, Cell Syst 3:133-143; Venayak N, Anesiadis N, Cluett W R, Mahadevan R. 2015, Engineering metabolism through dynamic control, Curr Opin Biotechnol 34:142-152; and Lalwani M A, Zhao E M, Avalos J L. 2018, Current and future modalities of dynamic control in metabolic engineering, Curr Opin Biotechnol 52:56-65.

As mentioned above, most synthetic circuits are brittle from an evolutionary point of view, in the sense that the host strain carrying the circuit is counter-selected by the environment in which it has to function (Renda B A, Hammerling M J, Barrick J E. 2014, Engineering reduced evolutionary potential for synthetic biology, Mol Biosyst 10:1668-1678). This is particularly so in the case of the growth switch, where a mutation relieving the repression of the genes encoding the RNA polymerase ββ′-subunits would lead the growing mutant bacteria to quickly overtake the growth-arrested population. The growth switch design does include safeguards, in particular the redundancy of the genes coding for the repressor Lacl (cf. EP 3 047 031). When moving to high-density cultures in large-scale bioreactors, however, one expects deleterious mutants to occur at a higher frequency and these safeguards may not be sufficient.

Although several strategies for increasing the genetic reliability of synthetic circuits have been proposed in the literature, there is room for improvement.

The embodiments according to the present invention drastically improve the synthetic circuit in such a way as to increase the genetic stability of the production strain. For this, the invention provides a genetic architecture such that single mutations do not affect its functioning (also called: mutational robustness). In particular, the genetic architecture of the microorganism of the invention introduces redundancy in the expression control of RNA polymerase.

More precisely, according to a first embodiment, the Applicant has put an additional subunit of RNA polymerase, the α subunit, under the control of the same or (preferably) different induction system(s) as used for controlling the expression of the ββ′-subunits. In other words, the genes encoding the ββ′-subunits are under the control of a first inducible promoter, while the gene encoding the α subunit is under the control of a second inducible promoter. As a consequence, only simultaneous mutations in both promoter regions upstream of the genes coding for a and ββ′ will disable expression control of RNA polymerase, and therefore growth control. In practice, this is a very unlikely event, in that the theoretical probability that a mutation disables the modified growth switch is the square of the mutation probability of the original genetic circuit. The growth switch with redundant control of RNA polymerase expression is thus genetically more stable.

In order to obtain genetically stable bacteria according to the first embodiment, a number of criteria must be met regarding the displacement of the gene coding for the α subunit.

First, the gene coding for the α subunit must be operably linked to a second inducible promoter (replacing the natural promoter of the gene). Furthermore, the gene coding for the α subunit must be inserted by homologous recombination into a locus constituted of genes that are non-essential for the growth of the bacteria or into a non-coding sequence, and at a maximum distance of 500,000 nucleotides from the replication origin. In addition, the gene sequence chosen for insertion of the gene encoding the α subunit operably linked to the second inducible promoter must not be susceptible to spontaneous homologous recombination. The gene coding for the α subunit must also be oriented in the opposite direction of the upstream gene (i.e. the gene upstream of the promoter of the gene coding for the α subunit). Lastly, the gene encoding the α subunit is deleted from its natural site. More precisely, the natural gene encoding the α subunit may simply be deleted, or it may correspond to the gene encoding the α subunit that is displaced according to the above-mentioned conditions, but the bacterium must not include a copy in the natural site of the gene coding for this α subunit. It was observed that when one or more of these criteria were omitted, the genetically modified bacteria obtained were not sufficiently stable.

For instance, the gene coding for the α subunit may be inserted by homologous recombination into one of the following insertion sites: insG, yjhD, insO-ins/1-insM, insA-insB1, ryjB, yjhR, envZ-ompR, or arcZ. Their functions, which are not essential to the growth of the bacteria in common biotechnological production conditions, are described in the following table:

TABLE 1
alternative insertion sites for the gene encoding
α subunit satisfying the criteria for insertion

| Insertion site | Annotation |
|---|---|
| insG | Coding for transposase |
| yjhD | No identified role |
| insO-insI1-insM | Pseudo-gene |
| insA-insB1 | No identified role |
| ryjB | Short non-coding sequence |
| yjhR | Pseudo-gene |
| envZ-ompR | Coding for kinase-regulator |
| | pair, non-essential |
| arcZ | Short non-coding sequence |

Accordingly, the method of this first embodiment comprises a step of bacterial growth wherein the expression of the genes encoding the ββ′ subunits and the α subunit is induced, i.e. the expression of rpoB, rpoC and rpoA. This leads to production of RNA polymerase within the cells. During this step bacterial growth takes place and the metabolite, peptide or recombinant protein is naturally produced in a natural amount. The method then comprises a further step wherein the expression of the genes encoding the ββ′ subunits and the α subunit is inhibited. Without the synthesis of new subunits, the cellular concentration of RNA polymerase decreases to a level below which it can no longer support bacterial growth. However, since the enzymes of the relevant metabolic pathway are sufficiently expressed and are stable, they remain present after growth arrest and the production of the metabolite continues. Moreover, even in the presence of low concentrations of RNA polymerase, the expression of peptides or recombinant proteins can continue.

The above method will be reinforced when the expression of the enzymes of the metabolic pathway or the expression of the peptide or homologous protein is put under the transcriptional control of a strong promoter. In fact, the remaining amounts of RNA polymerase within the cells will preferentially transcribe this strong promoter. A strong promoter is a promoter whose strength is at least 10% of that of a so called rrn promoter, cf. Liang et al. (1999), J. Mol. Biol. 292:19-37.

According to a second embodiment, the Applicant has put one or more genes encoding one or more small regulatory RNA(s) inhibiting the translation of messenger RNAs from the genes encoding the ββ′ subunits of RNA polymerase under the control of another induction system. The latter induction system is different from the induction system used for controlling the expression of the ββ′-subunits. The small regulatory RNA used in this embodiment aims to inhibit the synthesis of ββ′ subunits of RNA polymerase. Thus, only simultaneous mutations in both promoter regions upstream of the genes coding for ββ′ and small regulatory RNA will disable expression control of RNA polymerase, and therefore growth control.

In this second embodiment, there may be one or more srRNAs targeting β or β′ subunits. For example, it is possible to produce a modified bacterium with an srRNA against the β subunit and an srRNA against the β′ subunit. When only one type of srRNA is produced, it is preferentially chosen an srRNA designed to inhibit translation of the messenger RNAs from the gene encoding the β subunit.

Accordingly, the method of this second embodiment according to the invention comprises a step of bacterial growth control wherein the expression of the genes encoding the ββ′ subunits is induced, i.e. the expression of rpoB, rpoC, while the expression of the gene encoding the small regulatory RNA is inhibited. This leads to production of RNA polymerase within the cells. During this step bacterial growth takes place and the metabolite, peptide or recombinant protein is naturally produced in a natural amount. The method then comprises a further step wherein the expression of the genes encoding the ββ′ subunits is inhibited, while the expression of the genes encoding the small regulatory RNA is induced so as to inhibit the translation of potential mRNAs of the ββ′ subunits. Thus, the inhibition of the expression of the genes encoding the ββ′ subunits is reinforced. Without the synthesis of new subunits, the cellular concentration of RNA polymerase decreases to a level below which it can no longer support bacterial growth. However, since the enzymes of the relevant metabolic pathway are sufficiently expressed and are stable, they remain present after growth arrest and the production of the metabolite continues. Moreover, even in the presence of low concentrations of RNA polymerase, the expression of peptides or recombinant proteins can continue.

The expression of the genes encoding the enzymes producing the metabolite, or the expression of the peptide or the recombinant protein of interest can be further improved by the addition of a second polymerase having catalytic subunits that are different from the ββ′ subunits and/or α subunit of the host RNA polymerase operably linked to the inducible promoter. This second RNA polymerase can specifically increase transcription of the genes encoding the target enzymes producing the metabolite, or specifically increase transcription of the peptide or recombinant protein. However, the presence of such a second RNA polymerase is not essential. Indeed, the Applicant integrated a fluorescent reporter gene (mCherry under the control of the rrnBp2 promoter) into the genome of the E. coli strain IJ40. The results showed that the average fluorescence per cell increases after growth arrest, from 80 to 120 relative fluorescence units. This means that the growth-arrested cells synthesize proteins and are therefore metabolically active.

To carry out the method of the first embodiment of the invention, the Applicant designed a bacterium in which inducible promoters are used to regulate the expression of the rpoBC operon and the rpoA operon, which respectively encode the ββ′-subunits and the α-subunit of RNA polymerase Whereas, in the second embodiment, the Applicant designed a bacterium in which inducible promoters are used to regulate the expression of the rpoBC operon and the operon encoding the small regulatory RNA inhibiting the translation of messenger RNAs from the genes encoding the ββ′ subunits. With the bacterium (or bacterial strain) of the present solutions the intracellular concentration of RNA polymerase can be externally set to a desired level. As a consequence, the expression of the bacterial genes can be controlled.

The inducible promoters are of the same type or are different depending on the embodiment and/or the desired control of gene expression.

In particular, for the first embodiment, two identical promoters are preferably used in order to simultaneously control the expression of the genes coding for the ββ′ and α subunits of the RNA polymerase.

Conversely, with regard to the second embodiment, two different promoters are used in order to distinctly control the expression of the genes encoding the ββ′ subunits of RNA polymerase and small regulatory RNA inhibiting the translation of messenger RNAs from the genes encoding the ββ′ subunits.

In these embodiments, an inducible promoter can be, for example a lactose or β-D-1-thiogalactopyranoside (IPTG)-dependent promoter, variants of the endogenous lac promoter. By removing IPTG (or lactose) from the culture medium, cell growth can be switched off, or at least drastically turned down from the rate of wild-type bacteria to a very low growth rate. The remaining cell growth in the absence of IPTG in the medium is due to remaining RNA polymerases molecules present in the bacterium before decreasing the IPTG concentration. By increasing the IPTG (or lactose) concentration in the culture medium, cell growth can be switched on again, in order to regenerate or further increase the size of the bacterial population.

Other chemically-regulated promoters, such as the tet promoter or araBAD promoter, or physically-regulated promoters, such as light or temperature dependent promoters, may be used for the solution. Such promoters are well known by the skilled person; cf. Terpe (2006), Appl. Microbiol Biotechnol, 72:211-222.

As discussed above the present solution is an improvement based on the method and engineered bacteria described in EP 3 047 031 (originally filed as PCT/EP2014/069719, published under WO 2015/036622, based on priority EP13306266). Accordingly, the terminology of the present description is well known by the skilled person. Further details regarding specific definitions used herein are given in EP 3 047 031, which shall be consulted by the reader.

FIG. 1 shows a conceptual view of an embodiment according to the invention. The molecular components on the right-hand side of FIG. 1, concerning the IPTG-dependent regulation of rpoBC, were already present in the previous version of the growth switch disclosed in EP 3 047 031. The components on the left-hand side are drastically improving the known growth switch, in particular regarding robustness and stability of the genetic system. More precisely, the components on the left-hand side concern the regulation of rpoA by the same IPTG-dependent regulatory mechanism as the one for rpoBC regulation. The mutational robustness of the system is obtained by the fact that any mutation in only one of the two promoter regions (rpoBC or rpoA) does not lead to the loss of growth control, while the occurrence of simultaneous mutations in both promoter regions is very unlikely. This robustness measure adds to the safeguard already included in the original design, namely the presence of three lac/copies on the genome, which protects against single mutations disabling Lac binding to the promoter.

More precisely, FIG. 1 shows the architecture of the synthetic circuit controlling RNA polymerase expression according to a preferred embodiment. The genes rpoBC, encoding the ββ′-subunits, and the gene rpoA, encoding the α subunit, are put under the control of the Lac repressor by replacing the natural promoters by a synthetic promoter. Addition of isopropyl β-D-1-thiogalactopyranoside (IPTG) to the growth medium relieves Lac repression and enables growth, whereas in the absence of IPTG, the rpoA and rpoBC genes are not expressed, and hence RNA polymerase is not produced.

The α subunit is present in two copies per molecule of RNA polymerase: α^{I} and α^{II}, also referred to as α₂. Each α subunit contains two domains: αNTD (N-Terminal domain) and αCTD (C-terminal domain). αNTD contains determinants for the assembly of RNA polymerase. αCTD contains determinants for the interaction with promoter DNA, i.e. the α subunit C-terminal domain recognizes upstream promoter elements.

The α subunit is produced in excess during cell growth. Given the abundancy of the α-subunit of RNA polymerase the results of the embodiment according to the invention are absolutely surprising in that a control switch located in the rpoA area can radically increase the genetic stability of the known control switch while preserving its metabolic production capacity. The genetic stability combined with the high-throughput metabolic production capacity of the invention is a drastic improvement compared to the state of the art systems.

FIG. 2 shows the genetic construction for IPTG-dependent control of rpoBC and rpoA transcription of a variant according to the invention.

FIG. 2A shows the control of rpoBC expression via the IPTG-inducible T5 promoter (as disclosed and discussed in EP 3 047 031). More particularly, the rpoBC upstream sequence was modified by inserting the IPTG-inducible T5 promoter (T5p) with two lacO binding sites and a selection marker with the spectinomycin resistance gene and its promoter spcRp. Inserting this sequence by homologous recombination allows the regulation of rpoBC expression by adjusting the concentration of IPTG in the medium (cf. Lutz R, Bujard H. 1997, Independent and tight regulation of transcriptional units in Escherichia coli via the LacR/O, the TetR/O and AraC/I1-12 regulatory elements, Nucleic Acids Res 25:2003-20). In absence of the inducer, the Lac protein binds to the lacO sites and prevents RNA polymerase binding.

FIG. 2B shows the control of rpoA expression via the same regulatory system. It shows the realization of the genetic control of rpoA of an embodiment according to the invention on the sequence level. The RNA subunit a is encoded by the rpoA gene. The original rpoA locus on the E. coli genome was modified by homologous recombination (Sharan, S. K., Thomason, L. C., Kuznetsov, S. G., and Court, D. L. (2009). Recombineering: a homologous recombination-based method of genetic engineering. Nat Protoc 4, 206-223.), replacing the coding region of rpoA by the coding region of a selection marker conferring chloramphenicol resistance (cmR). The introduction of the gene coding for the resistance to chloramphenicol provides an ideal selection of the successful recombinants. According to a variant of the invention, the rpoA gene, including the inducible promoter, was displaced from its natural location on the chromosome to another location, the sokA-insKJ-hokA sequence. This sequence was chosen because it is at approximately the same distance from the origin of replication as the rpoBC operon. The sokA-insKJ-hokA genes are not essential for the growth of the bacterium and were removed by the cloning procedure. According to a variant of the invention, both the rpoBC and the rpoA expression can be controlled by adjusting the IPTG concentration in the medium.

FIG. 2C shows the location of the genes on the chromosome of E. coli. It summarizes the resulting changes on the E. coli chromosome due to the above modifications.

FIG. 3 shows a sequence according to a variant of the invention wherein rpoBC is under the control of an IPTG-dependent promoter. All promoter elements and genes are indicated.

FIG. 4 and FIG. 5 show a sequence according to a variant of the invention wherein rpoA is replaced by Cm in the operon of ribosomal proteins starting with rpsM. This variant is made to avoid expression of rpoA under natural conditions and confer chloramphenicol resistance (cmR). All relevant genes are annoted in the figure according to standard nomenclature.

FIG. 6, FIG. 7, FIG. 8 and FIG. 9 show a sequence according to a variant of the invention wherein rpoA, which is under the control of an IPTG-dependent promoter, is inserted at the position and in replacement of the sokA-insKJ-hokA sequence. All relevant genes are annoted in the figure according to standard nomenclature.

Accordingly, a variant of the bacterium (or bacterial strain) of the present invention comprises a nucleotide sequence having at least 80%, preferentially 90%, and more preferentially 100% identity to the sequence of FIG. 3 (SEQ ID: 1).

Another variant of the bacterium of the present invention comprises a nucleotide sequence having at least 80%, preferentially 90%, and more preferentially 100% identity to the sequence of FIGS. 4 and 5 (SEQ ID: 2).

Another variant of the bacterium of the present invention comprises a nucleotide sequence having at least 80%, preferentially 90%, and more preferentially 100% identity to the sequence of FIGS. 6, 7, 8 and 9 (SEQ ID: 3).

Preferentially, another variant of the bacterium of the present invention comprises a nucleotide sequence having at least 80%, preferentially 90%, and more preferentially 100% identity to the sequences of FIGS. 3 to 9 (SEQ ID: 1, SEQ ID: 2 and SEQ ID: 3).

The procedure for increasing production yields in bioreactors using the new strain is the same as for the previous strain, cf. EP 3 047 031. Tests have shown that the new strain preserves the ability to switch on and off growth by supplying and removing IPTG from the medium, respectively. The results also demonstrate that the production yield of glycerol from glucose after growth arrest is preserved. Moreover, the results show that the genetic stability of the improved strain is higher than that of the original strain.

FIG. 10 shows the increased genetic stability of the strain with control of the expression of the ββ′ subunits and the α subunit in comparison with control of the expression of the ββ′ subunits only.

In FIG. 10A the strain, in which the expression of the rpoBC genes is under the control of an IPTG-inducible promoter, was precultured in M9 minimal medium with 0.2% glucose supplemented with IPTG (250 μM) in 40 biological replicates. Subsequently, each replicate culture was washed, diluted, and resuspended in the same medium without IPTG, thus preventing the synthesis of new ββ′ subunits of RNA polymerase. As a control, three replicates were washed, diluted, and resuspended in the same medium with IPTG. Starting from a low optical density of 0.005, the growth was then monitored at 24 h and 96 h after the start of the culture. While none of the cultures had escaped after 24 h, 33 out of 40 cultures escaped after 96 h, as indicated by an optical density close to the optical density of the controls.

FIG. 10B shows the same for the strain in which both the expression of the rpoBC genes and the rpoA gene is under the control of an IPTG-inducible promoter. None of the cultures escaped after 96 h. This proves a drastically higher genetic stability of the strain with double expression control according to an embodiment of the invention.

FIG. 11 shows growth, glucose consumption, and glycerol production of the wild-type strain and both the strains with simple and double control of RNA polymerase expression.

In FIG. 11A the wild-type strain, transformed with a plasmid allowing overexpression of enzymes responsible for glycerol synthesis, was grown in M9 minimal medium with 0.2% glucose in three biological replicates. The optical density (OD600) was measured at different time-points during the experiment (blue dots, i.e. dots relative to x). The same measurements, in the same conditions, were carried out for the strain in which the expression of the rpoBC genes is under the control of an IPTG-inducible promoter (red dots, i.e. dots relative to +) and the strain in which both the expression of the rpoBC genes and the rpoA gene is under the control of an IPTG-inducible promoter (yellow dots, i.e. dots relative to −). Contrary to the wild-type strain, the latter two strains stop growing after inoculation, because the medium contains no IPTG.

FIG. 11B shows Measurement of the glucose concentration in the medium. The strains with IPTG-dependent control of RNA polymerase expression continue to consume glucose after growth arrest.

FIG. 11C shows measurement of the glycerol concentration in the medium. Whereas the wild-type strain produces only a small amount of glycerol, which is consumed after the depletion of glucose, the growth-arrested strains convert glucose into glycerol at much higher levels. This supports the claim that inhibiting the expression of RNA polymerase prevents growth of the population and allows the reallocation of metabolic fluxes toward the biosynthesis of a metabolite of interest (glycerol in this case) at a high yield.

The Applicant noted that a genetically stable bacteria could not be obtained if the insertion parameters of the first embodiment (mentioned above) were not respected. In other words, simply moving the gene coding the α subunit of the RNA polymerase is not enough to obtain a genetically modified bacterium with improved stability. This was demonstrated by performing homologous recombination of the inducible gene encoding the α subunit into a site not complying the criteria of insertion of the first embodiment.

For testing purposes, bacteria comprising an inducible rpoA gene of the RNA polymerase inserted by homologous recombination into the insertion site yhiM-yhiN are designed, as shown in the scheme of FIG. 12A, while the natural rpoA gene is deleted. The corresponding modified bacteria have the genes coding for the ββ′ subunits of the RNA polymerase operably linked to another inducible promoter, as explained above. In addition, the chosen site is less than 500,000 nt from the origin of replication. However, the region yhiM-yhiN is susceptible to natural homologous recombination. Therefore, not all the predefined insertion criteria are met.

FIG. 12B shows the agarose gel migration results (30 minutes, 75V) for fragments obtained under various PCR (Polymerase Chain Reaction) conditions using the primers shown in FIG. 12A. In particular, the PCR are realized before and after recombination. The primers 1 and 2 are used to verify whether the genomic region is present before and after recombination, in eight different PCR conditions.

Before recombination, the region flanked by the primers and containing the cloning cassette is present (positive control in gel, around 2500 bp). Whereas, after recombination, the region expected to contain the inducible rpoA sequence does not appeared (absence of spot around 1400 bp). The Applicant is of the opinion that this attempt is unsuccessful because not all insertion criteria are met. In particular, the chosen region is prone to natural recombination or the insertion of transposons.

These results show that the construct shown in FIG. 12A does not provide genetically modified bacteria with genetic stability. Thus, these results reveal the complexity of obtaining genetically modified bacteria with improved stability.

FIG. 13 shows a conceptual view of the second embodiment according to the invention. The molecular components on the left-hand side of FIG. 13, concerning the IPTG-dependent regulation of rpoBC, as described above. The components on the right-hand side concern the regulation of srRNA (inhibiting the translation of rpoBC mRNAs) by an aTc-dependent regulatory (derivative of tetracycline). The mutational robustness of the system is obtained by the fact that any mutation in only one of the two promoter regions (upstream of rpoBC or srRNA) does not lead to the loss of growth control, while the occurrence of simultaneous mutations in both promoter regions is very unlikely.

More precisely, FIG. 13 shows the architecture of the synthetic circuit controlling RNA polymerase expression according to the second embodiment. The genes rpoBC, encoding the ββ′-subunits, and the gene encoding srRNAs are respectively put under the control of the Lac repressor and TetR repressor by replacing the natural promoters by synthetic promoters. Addition of isopropyl β-D-1-thiogalactopyranoside (IPTG) to the growth medium relieves Lac repression and enables the production of mRNAs and then ββ′-subunits of the RNA polymerase, whereas in the absence of IPTG, the rpoBC genes are not expressed, and hence RNA polymerase is not produced.

On the contrary, the addition of aTc to the medium relieves TetR repression and does not permit the translation of the mRNA ββ′-subunits of the RNA polymerase and thus does not permit growth. Whereas in the absence of aTc in the medium, the mRNAs of the ββ′-subunits are translated, and hence RNA polymerase is produced.

Thus, the dual control of these two induction circuits allows dual control of RNA polymerase production, and therefore of bacterial growth. In particular, the addition of the control of the translation of the mRNA of the ββ′-subunits allows to enforce the inhibition of the synthesis of RNA polymerase. Thus, only a highly improbable double mutation of the two promoters would lead to the production of the RNA polymerase and growth of the bacteria.

As known by the skilled person, in bacteria, the core RNA polymerase enzyme is generally composed of five subunits: β, β′, α′, α″ and ω, whereby the last subunit, w, can often be removed without adverse effects. It is further known to the skilled person that bacterial RNA polymerase architecture is universal. This is discussed, for example, in the review from the authors Borukhov and Nudler, RNA polymerase: the vehicle of transcription, Trends in Microbiol., 2008, 16, 126-134. This review states that “The catalytically competent core (subunit composition 2a, β′, P and ω) has been evolutionarily conserved in terms of its primary sequence, ternary structure and function”, cf. p. 126 of the review. This means that the transcriptional behavior, which is directly dependent on that catalytically competent core, is also conserved throughout bacterial species. As a consequence, the genetically engineered bacterium and the method of the embodiment of the invention work in E. coli or other bacteria, e.g. Bacillus subtilis, Bacillus thuringiensis, Lactobacillus fermentum, Synechocystis sp. PCC6803, Corynebacterium glutanicum, Deinococcus radiodurans.

Further variants of the invention include a bacterium or a method according to the embodiments of the invention:
wherein the genes encoding the ββ′ subunits of RNA polymerase are the rpoBC genes of the rpoBC operon;
wherein said gene encoding the α subunit of RNA polymerase is the rpoA gene of the rpoA operon.

The methods of the invention may comprise particular variants, combined or not, such as:
a variant wherein the bacteria are cultured in step a. until a population density comprised from 0.1 to 100 OD₆₀₀ is reached;
a variant comprising a sub-step a1. between steps a. and b. consisting in harvesting and preferentially washing the bacteria cultured in step a. and transferring them into the second culture medium of step b.;
a variant comprising measuring the metabolic activity of the cultured bacteria during step b.;
a variant wherein step c. is carried out when the metabolic activity measured during step b. decreases;
a variant wherein said first culture medium of step a. comprises M9 minimal medium supplemented with 0.4% glucose and 0.5 mM IPTG (or other inducers, such as lactose) and said second culture medium of step b. comprises M9 minimal medium supplemented with 0.4% glucose and is free of IPTG;
a variant wherein said first culture medium of step a. comprises M9 minimal medium supplemented with 0.2% glucose and 0.25 mM IPTG (2.5 μL IPTG 100 mM in 1 mL of M9) and said second culture medium of step b. comprises M9 minimal medium supplemented with 0.2% glucose and is free of IPTG;
a variant comprising a step pre-a. before step a. consisting in providing bacteria comprising: (1) a gene encoding said recombinant protein or at least one gene encoding an enzyme involved in the production of said peptide or metabolite, and (2) genes encoding the ββ′ subunits and a gene encoding the α subunit of a RNA polymerase operably linked to an inducible promoter. The particular inducible promoter could involve chemicals other than IPTG or even different frequencies of light (optogenetics).

1. A genetically engineered bacterium for producing at least one metabolite, peptide or recombinant protein comprising a gene encoding a recombinant protein of interest or at least one gene encoding an enzyme involved in the production of a peptide or metabolite of interest, and further comprising genes encoding the ββ′ subunits of an RNA polymerase operably linked to an first inducible promoter, the natural promoter of said genes being replaced by said first inducible promoter, characterized in that:
either it further comprises a gene encoding the α subunit of the RNA polymerase having the following features listed below:
said gene being operably linked to a second inducible promoter, the natural promoter of the gene being replaced by said second inducible promoter,
said gene being inserted by homologous recombination into a locus constituted of genes that are non-essential for the growth of the bacteria or into a non-coding sequence and said locus
being a gene sequence whose probability of being the subject of spontaneous natural homologous recombination is less than 30%, preferably less than 20%, even more preferably less than 10%,
said gene encoding the α subunit of the RNA polymerase being positioned at a maximum distance of 500,000 nucleotides from the replication origin, and
said gene being oriented in the opposite direction of the upstream gene of the promoter of the gene coding for the α subunit,
and the natural gene encoding the α subunit of the RNA polymerase is deleted from its natural location.

2. The genetically engineered bacterium according to claim 1, wherein said first inducible promoter and said second inducible promoter are two distinct types of inducible promoters.

3. The genetically engineered bacterium according to claim 1, wherein at least one of said inducible promoters depend on two distinct external inducer molecules.

4. The genetically engineered bacterium according to claim 1, wherein at least one of said inducible promoters is selected from the following promoters: IPTG-dependent promoter, arabinose-dependent promoter, rhamnose-dependent promoter.

5. The genetically engineered bacterium according to claim 1, wherein said genes encoding the ββ′ subunits of RNA polymerase are the rpoBC genes of the rpoBC operon and said gene encoding the α subunit of RNA polymerase is the rpoA gene of the rpsM operon.

6. The genetically engineered bacterium according to claim 1, wherein the bacterium is of the species Escherichia coli.

7. The genetically engineered bacterium according to claim 5, wherein the original rpoA locus is replaced by a selection marker, preferentially a selection marker conferring chloramphenicol resistance (cmR), by homologous recombination.

8. The genetically engineered bacterium according to claim 1, wherein said bacterium comprise at least three copies of the lacI gene.

9. The genetically engineered bacterium according to claim 1 comprising a nucleotide sequence having at least 80%, preferentially 90%, and more preferentially 100% identity to at least one nucleotide sequence from the group consisting of SEQ ID NO: 1, SEQ ID NO: 2 and SEQ ID NO: 3.

10. The genetically engineered bacterium according to claim 1 comprising a nucleotide sequence having at least 80%, preferentially 90%, and more preferentially 100% identity to SEQ ID: 1, SEQ ID: 2 and SEQ ID: 3.

11. A method for producing at least one metabolite, peptide or recombinant protein of interest comprising the steps of
a. culturing bacteria comprising:
(i) a gene encoding said recombinant protein or at least one gene encoding an enzyme involved in the production of said peptide or metabolite,
(ii) genes encoding the ββ′ subunits of bacterial RNA polymerase operably linked to a first inducible promoter, the natural promoter of said genes being replaced by said first inducible promoter, and
(iii) a gene encoding the α subunit of bacterial RNA polymerase having the following features listed below:
said gene being operably linked to a second inducible promoter, the natural promoter of the gene being replaced by said second inducible promoter,
said gene being inserted by homologous recombination into a locus constituted of genes that are non-essential for the growth of the bacteria or into a non-coding sequence and said locus being a gene sequence whose probability of being the subject of spontaneous natural homologous recombination is less than 30%, preferably less than 20%, even more preferably less than 10%,
said gene encoding the α subunit of the RNA polymerase being positioned at a maximum distance of 500,000 nucleotides from the replication origin, and
said gene being oriented in the opposite direction of the upstream gene of the promoter of the gene coding for the α subunit,
and the natural gene encoding the α subunit of the RNA polymerase is deleted from its natural location, in a first culture medium inducing the expression of the genes encoding the ββ′ subunits and further inducing the expression of the gene encoding the α subunit, thereby inducing bacterial growth;
b. culturing said bacteria in a second culture medium inhibiting the expression of the genes encoding the ββ′ subunits and further inhibiting the expression of the gene encoding the α subunit, thereby producing said metabolite, peptide or recombinant protein while inhibiting bacterial growth;
c. optionally iterating steps a. and b. successively; and
d. optionally recovering said metabolite, peptide or recombinant protein produced by said bacteria.

12. The method according to claim 11, wherein each inducible promoter is an IPTG-dependent promoter or a lactose-dependent promoter.

13. The method according to claim 11, wherein the first culture medium of step a. comprises IPTG or lactose and the second culture medium of step b. is free of IPTG or lactose.

14. The method according to claim 12, wherein the gene encoding said recombinant protein or said at least one gene encoding an enzyme involved in the production of said peptide or metabolite is transcribed by a second RNA polymerase having a catalytic subunit or catalytic subunits that are different from the ββ′ subunits and the α subunit of the RNA polymerase operably linked to the inducible promoter.

15. The method according to claim 11, wherein said second RNA polymerase is the bacteriophage T7 polymerase.

16. The method according to claim 11, wherein said bacteria are Escherichia coli bacteria.

17. (canceled)
